(19) United States
(12) Patent Application Publication
(10) Pub. No.: US 2010/0120707 A1
(43) Pub. Date: May 13, 2010

(54) Dosages for menstrual suppression, contraception, and hormone replacement therapy, and methods of administering same

(75) Inventors: Eugene P. Dougherty, JR., Camden-Wyoming, DE (US); Michael Kalmon, Frederica, DE (US); Frank Glaug, Chester Springs, PA (US); Patrick Gorham, Camden-Wyoming, DE (US)

Correspondence Address: OHLANDT, GREELEY, RUGGIERO & PERLE, LLP, 1 LANDMARK SQUARE, 10TH FLOOR, STAMFORD, CT, 06901, US

(73) Assignee: Playtex Products, LLC

(21) Appl. No.: 12/586,038

(22) Filed: Sep. 16, 2009

Related U.S. Application Data
(60) Provisional application No. 61/192,158, filed on Sep. 16, 2008.

(51) Int. Cl. A61K 31/7008; A61K 31/65; A61K 31/565; A61P 5/00

(52) U.S. Cl. 514/29; 514/154; 514/170; 514/171

(57) ABSTRACT

A dosage comprising an antibiotic, an estrogen, and a progestagen is provided. The dosage can be oral, or administered in other methods, such as intravaginally with a tampon. The dosage can be used for menstrual suppression, contraception, and/or hormone replacement therapy. A method of treating a patient with the dosage comprises administering the dosage to the patient over a course of treatment, in amounts or frequencies that can remain constant, or be varied.

### CROSS-REFERENCE TO RELATED APPLICATIONS

The present application claims the benefit of U.S. Patent Application No. 61/192,158, filed on Sep. 16, 2008.

### BACKGROUND OF THE DISCLOSURE

1. Field of the Disclosure

The present disclosure relates to dosages for menstrual suppression, contraception, and/or hormone replacement therapy, and methods of administering the same. More particularly, the present disclosure relates to a dosage comprising an estrogen, progesterone, and an antibiotic, and a method of administering this dosage.

2. Description of the Related Art

Many women are interested in methods of menstrual suppression, contraception, and hormone replacement therapy. Menstrual suppression medicaments can assist with reducing the pain, number, and frequency of menstrual periods, and may also have some health benefits such as a reduced risk of certain cancers. Contraception can help with family planning. Hormone replacement therapy can have significant health benefits for older women, such as reducing “hot flashes,” and preventing osteoporosis.

It can be very difficult, however, to prepare a single drug, or dosage including a combination of drugs, that adequately addresses all three of these goals. Many drugs that are designed for one or more of these uses can have adverse interactions with those designed for the others. These adverse side effects can include abnormal bleeding, weight gain, bloating, acne, masculine characteristics, hair loss or growth, or others.

In addition, many women that use current dosages experience what is called “breakthrough bleeding.” These women, rather than having a predictable period, find that the dosage causes an unpredictable amount of vaginal bleeding. It may sometimes be minor, but can also be more significant, which can be very embarrassing and inconvenient to the user. The breakthrough bleeding can also continue long after the user has commenced taking the dosage. Instead of eliminating the need for tampons, this phenomenon requires an extra level of vigilance on the part of the user. Many users of these dosages consequently get discouraged.

Therefore, there is a need for a new dosage, and a method of using the same, that can successfully be used as a contraceptive, menstrual suppressant, and for hormone replacement therapy, that does not experience the drawbacks of currently available dosages.

### SUMMARY OF THE DISCLOSURE

In one embodiment, the present disclosure provides a dosage for use for menstrual suppression, contraception, and/or hormone replacement theory. The dosage comprises an antibiotic, and at least one of an estrogen and a progestagen.

In another embodiment, the present disclosure provides a dosage comprising an antibiotic, an estrogen, and a progestagen.

In another embodiment, the present disclosure provides methods of administering a dosage to a patient, wherein the dosage comprises an antibiotic, an estrogen, and a progestagen. The methods comprise the step of administering the dosage at a frequency, over a course of treatment.

### DETAILED DESCRIPTION OF THE DISCLOSURE

The present disclosure has surprisingly found that, contrary to commonly held beliefs, an antibiotic can be combined in a dosage with both an estrogen and a progestagen to provide safe and efficacious contraception, menstrual suppression, and/or hormone replacement therapy, all at once. Alternatively, the antibiotic could be combined in a dosage with either the estrogen or the progestagen alone. Previously, it was thought that antibiotics, such as those of the present disclosure, would have an adverse effect on either or both of the estrogens or progestagens, and thus limit their effectiveness in contraception, menstrual suppression, or hormone replacement therapy. The antibiotic has been found to address the problem of breakthrough bleeding. Dosages having all three of these components have not previously been developed.

1. Estrogens

The estrogens used in the dosage of the present disclosure can be anyone of the following: ethinyl estradiol, estradiol, estropipate, natural conjugated equine estrogens, conjugated synthetic estrogens, esterified estrogens, estrogen sulphamate, estrone sulfate, piperazine estrone sulfate, mestranol, estriol, estrone, estradiol valerate, dinestrol, or any combinations thereof. The amounts of estrogen in the present dosages can be about 0.005 mg to about 1 mg, or from about 0.01 mg to about 0.625 mg, or from about 0.02 to about 0.05 mg, or their equivalent amounts in liquid form, all on a per diem basis. The amounts of estrogen can also be precisely those amounts, i.e. 0.005 mg to 1 mg, or from 0.01 mg to 0.625 mg, or from 0.02 to 0.05 mg. It is believed that estrogen dosage amounts outside these ranges are not effective for all three of contraception, menstrual suppression, and hormone replacement therapy. Table 1 below shows dosage amounts that can be advantageous for specific estrogens from the list recited above.

TABLE 1

| | |
|---|---|
| Ethinyl Estradiol | about 0.02 mg-about 0.05 mg |
| Estradiol | about 1 mg |
| Estropipate | about 0.05 mg-about 0.15 mg |
| Natural conjugated equine estrogens | up to about 0.625 mg |
| Mestranol | about 0.05 mg |

Again, the amounts of the estrogens listed in Table 1 can be precisely the amounts listed, instead of “about” the amount. For example, estradiol can be present in the dosage in an amount of 1.0 mg.

2. Progestagens

The progestagen of the present disclosure can be the natural progestagen progesterone, or a synthetic progestagen, such as a progestin. Generally, the progestagen can be present in an amount of about 0.01 to about 7 mg, or the equivalent amount in a liquid dosage, per diem, or precisely that amount, i.e. 0.01 mg to 7 mg. Table 2 shows a list of possible progestagens that can be used in the dosages of the present disclosure, and their dosage amounts.

TABLE 2

| | |
|---|---|
| Levonorgestrel | about 0.05 mg-about 0.15 mg |
| Norethisterone | about 0.4 mg-about 1 mg |
| Norethindrone | about 0.05 mg-about 1 mg |
| Norethindrone acetate | about 1.0 mg-about 1.5 mg |
| Norgestimate | about 0.018 mg-about 0.250 mg |
| Norgestrel | about 0.3 mg-about 0.5 mg |
| Cyproterone | up to about 2 mg |
| Desogestrel | about 0.1 mg-about 0.15 mg |
| Drospirenone | about 0.5 mg to about 4 mg |
| Ethynodol acetate | up to about 1 mg |
| Gestodene | about 0.05 mg-about 0.1 mg |

The progestagens listed in Table 2 may be used singly, or in combinations of one or more. Other progestagens contemplated for the dosages of the present disclosure include cyperoterone, cyprotone acetate, ethynodiol diacetate, medroxyprogesterone, and noreldestromin, or combinations thereof, with the progestagens listed in Table 2. Furthermore, the amounts of the progestagens listed in Table 2 can be precisely the amounts listed, instead of “about” the amount. For example, levonorgestrel can be present in the dosage in an amount of 0.05 mg to 0.15 mg.

3. Antibiotics

The antibiotic of the present disclosure can be a tetracycline, such as doxycycline, doxycycline hydrochloride, demeclocycline, meclocycline sulfosalicyate, minocylcine hydrochloride, oxytetracycline, tetracycline hydrochloride, or any combinations thereof. Besides the aforementioned tetracycline-type antibiotics, penicillin, oxacillin, erythromycin, ciprofloxacin, methicillin, nafcillin, clindamycin, vancomysin, ampicillin, or any combinations thereof can also be used.

The dosage amount of some of these antibiotics has often been fairly high to treat infections. For example, in the prior art, oxytetracycline has been recommended at dosages of up to 2000 mg per diem, and tetracycline hydrochloride has been recommended at dosage levels of up to 2000 mg per diem. The present disclosure has discovered, however, that dosages of much smaller amounts can be used for menstrual suppression, contraception, and for hormone therapy, contrary to what is customary for these antibiotics. The amount of the antibiotic in the dosages of the present disclosure can be from about 1 mg to about 1000 mg, or about 2 mg to about 100 mg, or about 5 mg to about 20 mg, or the equivalent amounts in a liquid form, all on a per diem basis. The antibiotics can also be present in precisely those amounts, i.e. 1 mg to 1000 mg, or 2 mg to 100 mg, or 5 mg to 20 mg.

Doxycycline can be sold under a number of brand names, for example Adoxa, Atridox, Bio-Tb, Doryx, Doxy Caps, Doxycel Hyclate, Monodox, Periostat, Vibramycin, and Vibra-Tabs. Demeclocycline can be sold as Declomycin. Meclocycline sulfosalicyate can be sold as Meclan. Minocycline hydrochloride can be sold as Arestin, Dynacin, Minocin and Vectrin. Oxytetracycline can be sold as Terrramycin and Uri-Tet, and tetracycline hydrochloride can be sold as Achromycin V, Ala-Tet, Bristacycline, Nor-Tet, Panmycin, Sumycin, Tetracap, Tetracyn, Tetralan and Topicycline.

4. Dosage Forms

The dosage containing the three components discussed above can be a solid or liquid oral dosage, e.g., in a tablet, capsule, caplet, gel-cap, or drops. The dosage can also be administered via an implant, an injection, or transdermally, i.e. a “patch” or a spray. The dosage can also be delivered intravaginally, for example through use of a tampon. When in solid forms such as in a tablet, the estrogen, progestagen, and antibiotic can be in a micronized form, which speeds delivery and dissolution of these ingredients.

In one embodiment, the dosage comprises: about 0.02 mg to about 0.05 mg of ethinyl estradiol per day, about 2 mg to about 4 mg of drospirenone per day, and about 5 mg to about 20 mg of doxycycline hydrochloride, or the equivalent liquid amounts of these three compounds, per day.

In a second embodiment, the dosage comprises: about 0.005 mg to about 0.06 mg of ethinyl estradiol per day, about 0.1 mg to about 7 mg of drospirenone per day, and about 0.5 mg to about 100 mg of doxycycline, or the equivalent liquid amounts of these three compounds, per day.

In a third embodiment, the dosage comprises: about 0.02 mg of ethinyl estradiol per day, about 3 mg of drospirenone per day, and about 25 mg of doxycycline, or the equivalent liquid amounts of these three compounds, per day.

In any of the three embodiments discussed above, the ingredients can be present in precisely the amounts listed, i.e. without “about.” For example, the amount of the ethinyl estradiol in the third embodiment can be 0.02 mg.

The amounts of the three ingredients in the dosage may be varied throughout the course of treatment, on a day to day basis, or week to week, to achieve the proper balances of the ingredients in the user. The amounts of the ingredients can also be varied to target specific uses. For example, if contraception is the primary objective of the dosage, then the amount of antibiotic can be lowered. For hormone replacement therapy, the amount of the estrogen can be increased, while the antibiotic and progestagen can be lowered. For example, about 0.5 mg of drosperinone can be a typical dosage level of progestin used in menstrual suppression.

In one embodiment, the dosage can be administered once daily for twenty-one days, followed by seven days of a daily dosage of a placebo, or a sugar pill. This regimen may be better suited for contraception means. In another embodiment, the dosage can be administered once daily for an extended period of from twenty-eight days up to 365 days. This embodiment may be more suitable for menstrual suppression.

A method of using the dosage of the present disclosure for contraception, menstrual suppression, and hormone replacement therapy would encompass all of these dosage forms, amounts, and treatment schedules.

5. Additional Ingredients

The dosages of the present disclosure, in either solid or liquid form, may also contain other ingredients, such as one or more carriers, excipients, adjuvants, flavoring agents, coloring agents, preservatives, antioxidants, or any combinations thereof. Examples of these ingredients include:
carriers such as starch, sodium lauryl sulfate, Polysorbate 80, pre-gelatinized starch, nonionic surfactants, pharmaceutical glaze polyethylene glycol, carnauba wax, water, corn oil, sesame oil, and/or peanut oil;
excipients such as lactose, lactose monohydrate, microcrystalline cellulose, methylcellulose, ethylcellulose, hydroxypropylmethylcellulose, hydroxypropyl cellulose, sugar alcohols such as xylitol, sorbitol and/or maltitol;
adjuvants such as povidone;
glidants/lubricants, such as silica or stearic acid
coloring agents, pigments and food grade dyes;
opacifiers, such as titanium dioxide;
flavoring agents, such as sucrose, fructose, corn syrup, vanilla, mint, cherry, anise, peach, apricot, licorice, or raspberry;
antioxidants, such as vitamin A, vitamin C, vitamin E, retinyl palmitate, and/or selenium;
preservatives, such as citric acid, sodium citrate, propyl paraben
fillers such as dicalcium phosphate, calcium phosphate tribasic, calcium sulfate, and, triethyl citrate; and
anti-adherents such as magnesium stearate.

The present disclosure having been thus described with particular reference to certain embodiments thereof, it will be obvious that various changes and modifications may be made therein without departing from the spirit and scope of the present disclosure.

What is claimed is:

1. A dosage for use for menstrual suppression, contraception, and/or hormone replacement therapy, comprising:
an antibiotic; and
at least one of an estrogen and a progestagen.

2. A dosage, comprising:
an antibiotic;
an estrogen;
and a progestagen.

3. The dosage of claim 2, wherein said antibiotic is a tetracycline.

4. The dosage of claim 3, wherein said tetracycline is selected from the group consisting of doxycycline, doxycycline hydrochloride, demeclocycline, meclocycline sulfosalicyate, minocylcine hydrochloride, oxytetracycline, tetracycline hydrochloride, and any combinations thereof.

5. The dosage of claim 2, wherein said antibiotic is selected from the group consisting of penicillin, oxacillin, erythromycin, ciprofloxacin, methicillin, nafcillin, clindamycin, vancomysin, ampicillin, and any combinations thereof.

6. The dosage of claim 2, wherein said estrogen is selected from the group consisting of ethinyl estradiol, estradiol, estropipate, natural conjugated equine estrogens, conjugated synthetic estrogens, esterified estrogens, estrogen sulphamate, estrone sulfate, piperazine estrone sulfate, mestranol, estriol, estrone, estradiol valerate, dinestrol, and any combinations thereof.

7. The dosage of claim 2, wherein said progestagen is a natural progestagen.

8. The dosage of claim 7, wherein said natural progestagen is progesterone.

9. The dosage of claim 2, wherein the progestagen is a synthetic progestagen.

10. The dosage of claim 9, wherein said synthetic progestagen is selected form the group consisting of levonorgestrel, norethisterone, norethindrone, norethindrone acetate, norgestimate, norgestrel, cyproterone, desogestrel, drospirenone, ethynodol acetate, gestodene, cyperoterone, cyprotone acetate, ethynodiol diacetate, medroxyprogesterone, noreldestromin, and any combinations thereof.

11. The dosage of claim 2, wherein said antibiotic is present in an amount of 1 mg to 1000 mg.

12. The dosage of claim 2, wherein said estrogen is present in an amount of 0.005 mg to 1 mg.

13. The dosage of claim 2, wherein said progestagen is present in an amount of 0.01 mg to 7 mg.

14. The dosage of claim 2, wherein the dosage is a solid or liquid oral dosage form.

15. The dosage of claim 2, wherein the dosage is administered in an intravaginal form.

16. The dosage of claim 1,
wherein said antibiotic is doxycycline hydrochloride and is present in an amount of 0.5 mg to 100 mg,
wherein said estrogen is ethinyl estradiol and is present in an amount of 0.005 mg to 0.06 mg, and
wherein said progestagen is drospirenone and is present in an amount of 0.1 mg to 7 mg.

17. A method of administering the dosage of claim 2, the method comprising the step of:
administering the dosage at a frequency of at least once per day, over a course of treatment.

18. The method of claim 17, further comprising the step of varying said frequency, over said course of treatment.

19. The method of claim 19, wherein said dosage comprises an amount of each of said antibiotic, said estrogen, and said progestagen, and the method further comprises the step of:
adjusting at least one of said amounts of said antibiotic, said estrogen, and said progestagen, over said course of treatment.

20. The method of claim 17, wherein said dosage is administered for menstrual suppression.
